(19) United States
(12) Patent Application Publication
(10) Pub. No.: US 2006/0134773 A1
(43) Pub. Date: Jun. 22, 2006

(54) Device for pretreating specimen

(76) Inventors: Ken Inose, Kyoto (JP); Shinya Nakajima, Kyoto (JP); Satoshi Hashiguchi, Kyoto (JP); Jun Murakami, Kyoto (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/536,827

(22) PCT Filed: Nov. 27, 2003
(86) PCT No.: PCT/JP03/15133

§ 371 Date: Oct. 31, 2005

(30) Foreign Application Priority Data
Nov. 28, 2002 (JP) 2002-345211

(51) Int. Cl. C12M 1/34

(52) U.S. Cl. 435/287.1

(57) ABSTRACT

Due to the recent decipherment of the human genome, various relations between life processes and genes have been analyzed. Consequently, medical importance shifts from pathology to etiology, and from medical treatment to prevention. Hereupon, the gene test technology becomes an important basis. A pretreatment device for pretreating a specimen comprises a specimen introducing portion, a holding portion, a wash storage, an elute storage, and a discharging portion in order to prevent a loss of sensitivity during a pretreatment process of the nucleic acid test, to easily automate the pretreatment process, and to decrease the cost thereof, thereby serving as a familiar system for the gene test.

### BACKGROUND OF THE INVENTION

1. Field of the invention

The present invention relates to a device for pretreating a specimen, and in more detail, relates to a pretreatment device for extracting nucleic acids to be examined from biomass and the like in nucleic acid test.

2. Background Art

Due to the recent decipherment of the human genome, various relations between life processes and genes have been analyzed. Consequently, medical importance shifts from pathology to etiology, and from medical treatment to prevention. Hereupon, the gene test technology becomes an important basis.

The gene test can be used for tests which are difficult to be carried out by the conventional clinical examination, such as identification of pathogenic microorganisms difficult to be cultivated, detection of pathogenic microorganisms under medical treatment with antibiotics or at an early stage of infection, detection of antigens in case of suspect of existence of transferred antibody, investigation of a source of infection of pathogenic microorganisms, personal identification such as parentage diagnosis, gene diagnosis of disease type of leukemia and solid tumor, and established diagnosis of genetic disease. Bacteria, which requires a long time for cultivation thereof, can be effectively detected by the gene test, because the gene test takes a short time compared with a method using cultivation of bacteria. Furthermore, since DNA is stable depending on a good storage condition, an old sample, such as a frozen biopsy or a bone, also can be tested.

The gene test attracts public attention because it can expand opportunities of test of recently increasing sexual infectious diseases.

There are well-known conventional methods of purification and concentration of a nucleic acid, including a purification method using phenol, chloroform or ethanol, a purification method using a column which adsorbs nucleic acids, and a purification method using magnetic silica beads.

Furthermore, there is a well-known conventional method for recovery of a nucleic acid from a plate-like electrophoresis gel, as described in the Japanese Utility Model Laid Open Gazette Hei. 5-88296, whereby nucleic acids are electrophoresed in a prepared gel, a recovery device is moved to a portion of the gel involving target nucleic acids, and then the target nucleic acids are further electrophored to be recovered.

Furthermore, there is a well-known conventional method as described in the Japanese Patent Laid Open Gazette Hei. 8-327595, whereby nucleic acids are electrophoresed in a plate-like electrophoresis gel so as to liberate target nucleic acids, and a recovery chip is inserted into the gel near a band of the target nucleic acids so as to recover the target nucleic acids.

With regard to the conventional methods for purification and concentration of a nucleic acid, the purification method using phenol, chloroform or ethanol is available in only limited environments because it needs a powerful medicine requiring a highly advanced chemical equipment. Further, the purification method is difficult to be automated because it requires laborious operations and high-speed centrifugation process. It is also difficult to obtain high refining accuracy.

The purification method using a column for adsorbing nucleic acids is difficult to be automated because it requires centrifugation or aspiration process.

The purification method using magnetic silica beads is difficult to obtain high recovery of nucleic acid, because a silica bead, which is failed in recovery by a magnet or falls off from magnetic material, may remain in a sample.

The conventional method for recovering a nucleic acid from a plate-like electrophoresis gel requires the plate-like electrophoresis gel and the electrophoresis of nucleic acids in the plate-like electrophoresis gel before processing the portion of the gel involving the target nucleic acids.

The gel for electrophoresis is weak against a shock, and may change in characteristic according to formation process thereof. Therefore, generally, the position of the target nucleic acids in the electrophoresis gel is analyzed by ultraviolet rays after electrophoresis, and subsequently, the portion of the gel including high content of the target nucleic acids is processed.

Consequently, the gene test using this method takes a long time per unit of test. If the gel for electrophoresis is large, bleeding of the band of the nucleic acids is caused by unevenness of the gel, thereby reducing recovery of the nucleic acids. Furthermore, the large gel requires large electric power for the electrophoresis.

### DISCLOSURE OF THE INVENTION

In order to solve the above-mentioned problems, following means are used for the present invention.

A pretreatment device for pretreating a specimen comprises a specimen introducing portion, a holding portion, a wash storage, an elute storage, and a discharging portion.

The device combines a function for liberating nucleic acids off from a specimen containing the target nucleic acids is integrated with a function for extracting and purifying the liberated nucleic acids, thereby reducing a loss of sensitivity during a pretreatment process.

Accordingly, in the device, the nucleic acids can be liberated off from the specimen, and the liberated nucleic acids can be extracted and purified. The device can easily automate the pretreatment of specimen and decrease the cost. Additionally, the device can serve as a familiar system for the gene test.

### BRIEF DESCRIPTION OF THE DRAWINGS/FIGURES

FIG. 1 is a perspective view of a pretreatment device showing the whole construction thereof.

FIG. 2 is a perspective view of the pretreatment device showing assembly thereof.

FIG. 3 is a plane view of the device.

FIG. 4 is an arrow sectional view of the line A-A in FIG. 3.

FIG. 5 is an arrow sectional view of the line B-B in FIG. 3.

FIG. 6 is a plane view of the pretreatment device showing a process for holding nucleic acids.

FIG. 7 is a plane view of the pretreatment device showing a process for washing nucleic acids.

FIG. 8 is a plane view of the pretreatment device showing a process for eluting nucleic acids.

FIG. 9 is a plane view of a pretreatment device according to a second embodiment.

FIG. 10 is a plane view of a pretreatment device according to a third embodiment.

FIG. 11 is a plane view of a pretreatment device according to the third embodiment showing a process for extracting nucleic acids.

FIG. 12 is a view of a pretreatment device according to a fourth embodiment showing the construction thereof.

### BEST MODE FOR CARRYING OUT THE INVENTION

An embodiment of the present invention will be explained with reference to the accompanying drawings.

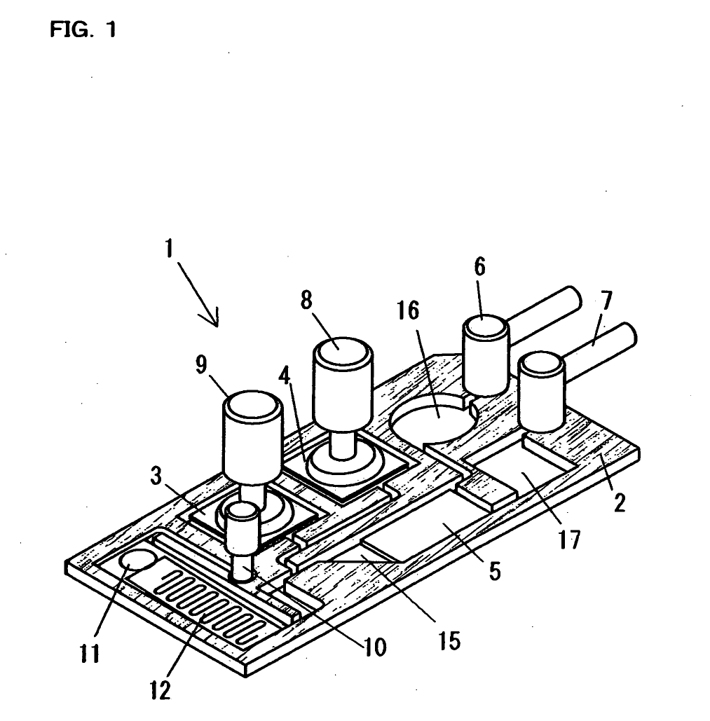

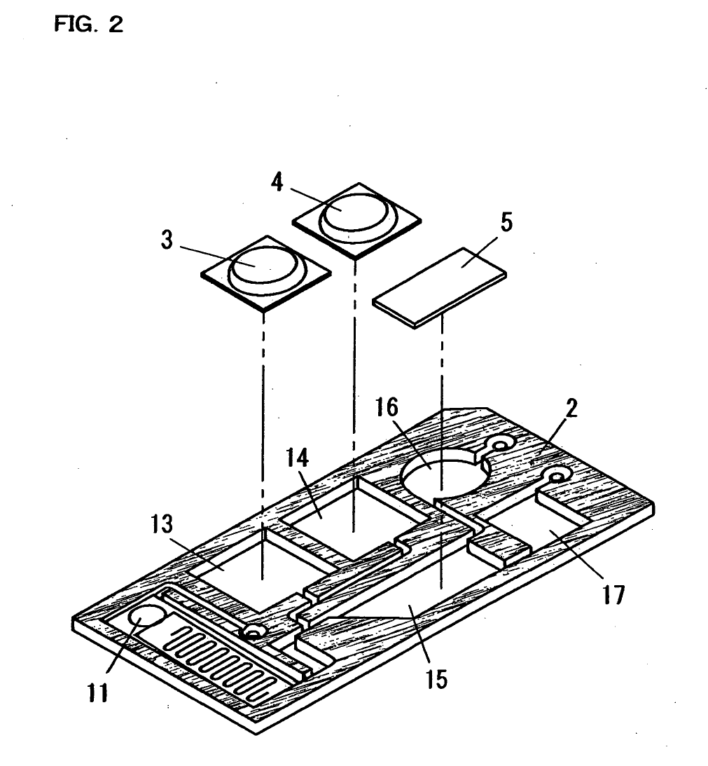

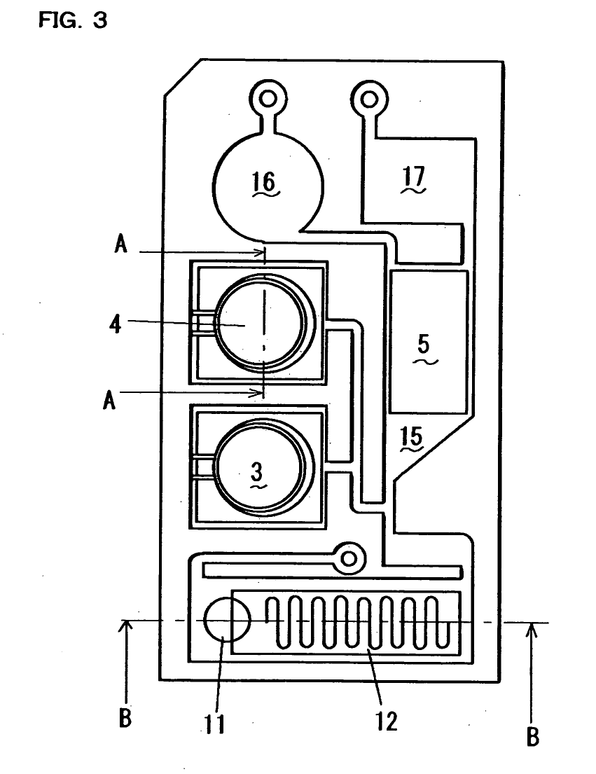

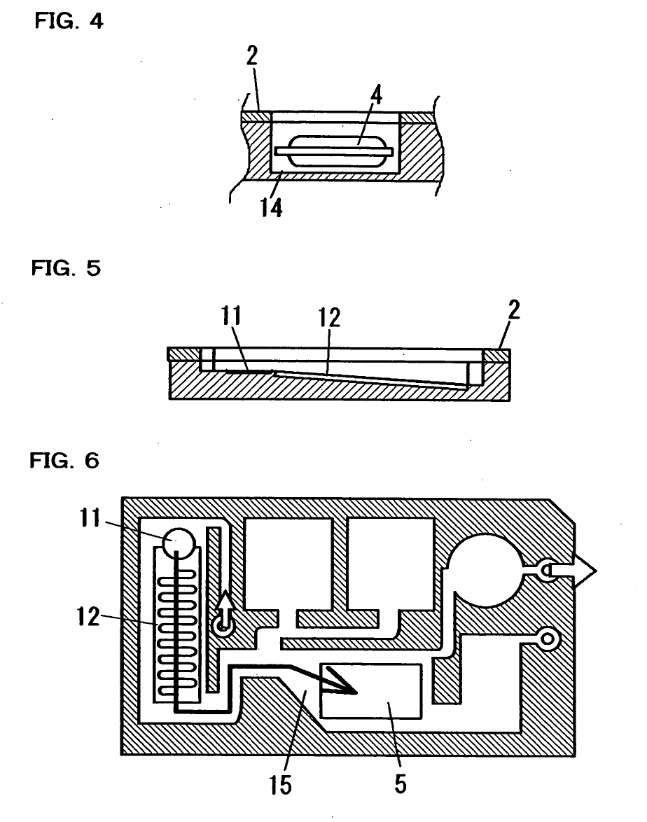

Explanation will be given of construction of a pretreatment device 1 with reference to FIGS. 1-5 inclusive.

In the pretreatment device 1, a specimen is introduced into an introducing portion 11, and nucleic acids are liberated off from the specimen. The nucleic acids are held in a holding portion 15 so as to be extracted after being washed. The pretreatment device 1 comprises the specimen introducing portion, the holding portion, a wash storage, a elute storage, and a discharging portion, which are formed in a base 2. The introducing portion 11 of the specimen, a heater 12 for liberating the nucleic acids off from biomass and virus, a holder 5 for holding the nucleic acids, a wash unit 3, and an elute unit 4 are provided on the base 2 of the pretreatment device 1. A valve 10, a connector 6, and a connector 7 are connected to grooves provided in the base 2. The connectors 6 and 7 connect the grooves of the base 2 with an air pump. Silica membrane and the like can be used as the holder 5.

Actuators 9 and 8 are disposed on the wash unit 3 and the elute unit 4, respectively. The actuators 9 and 8 are operated so as to push the wash unit 3 and the elute unit 4, thereby flowing a wash and an elute onto the base 2.

In the pretreatment device 1, the specimen is introduced into the introducing portion 11 and transmitted to the holding portion 15. In the base 2, as shown in FIG. 5, the heater 12 is provided in a falling slope which connects the introducing portion 11 and the groove leading to the holding portion 15. Accordingly, the specimen introduced into the introducing portion 11 is moved on the heater 12 by gravity, capillary phenomenon, or suction force of the pump 6 or 7. At this time, the heater 12 heats the specimen so as to liberate the nucleic acids therefrom.

The wash storage 13, where the wash unit 3 is disposed, and the elute storage 14, where the storage liquid unit 4 is disposed, are constructed as concave portions in the base 2. In the same way, concave portions of the base 2 are provided with the holding portion 15, the discharging portion 16, and an extracting portion 17, respectively. The holder 5 for adsorbing and holding the nucleic acids is provided in the holding portion 15. The connectors 6 and 7 leading to the air pump are connected with the discharging portion 16 and the extracting portion 17 through grooves.

Next, explanation will be given of the pretreatment by the pretreatment device with reference to FIGS. 6-8 inclusive. Firstly, the specimen is introduced into the introducing portion 11, and the nucleic acids are extracted from the specimen moving on the heater 12. The specimen is introduced into the holding portion 15 with the extracted nucleic acids, so that the nucleic acid is held by the holder 5. In this case, the valve 10 is opened, and then air is inhaled from the connector 6 connected with the discharging portion 16, thereby introducing the specimen into the holding portion 15 smoothly.

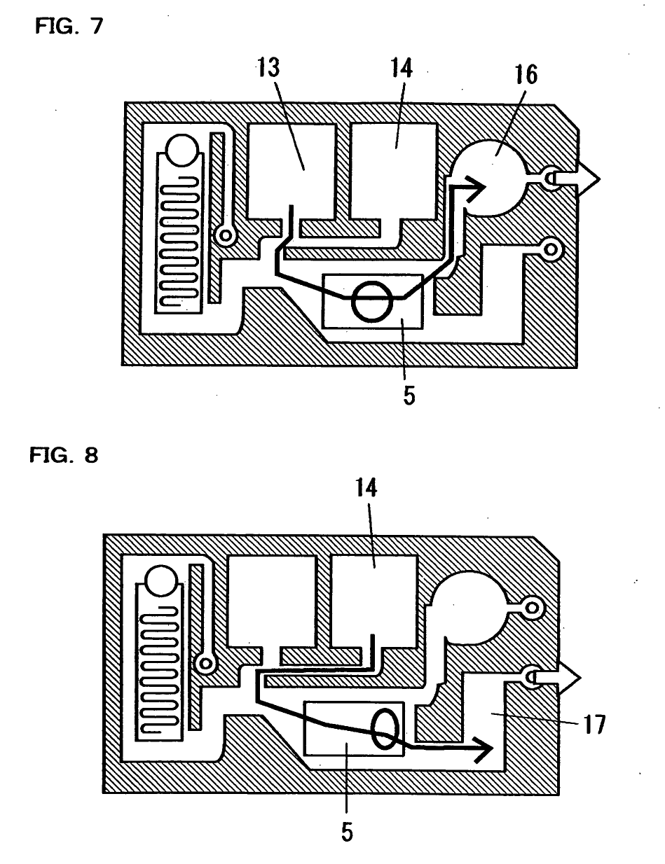

Subsequently, as shown in FIG. 7, the wash flows out from the wash storage 13 and washes the holder 15. The actuator 9 pushes the wash unit 3 so as to supply the wash from the wash storage 13 to the holding portion 15. The wash supplied to the holding portion 15 washes the holder 5, and then flows into the discharging portion 16. The nucleic acids are held by the holder 5, while unnecessary protein etc. flow into the discharging portion 16. In this case, the valve 10 is closed, and then air is inhaled from the connector 6 connected with the discharging portion 16, thereby introducing the wash into the holding portion 15 smoothly.

Next, as shown in FIG. 8, the elute flows out from the elute storage 14, and elutes the nucleic acids held in the holding portion 15. The actuator 8 pushes the elute unit 4 so as to supply the elute from the elute storage 14 to the holding portion 15. The elute supplied to the holding portion 15 elutes the nucleic acids which are absorbed to the holder 5, and then flows into the extracting portion 17. The held nucleic acids are released from the holder 5 so as to be supplied to the extracting portion. In this case, the valve 10 is closed, and then air is inhaled from the connector 7 connected with the extracting portion 17, thereby flowing the elute into the extracting portion 17 smoothly.

In this way, the single base 2 is provided with the specimen introducing portion 11, the holding portion 15, the wash storage 13, the elute storage 14, and the discharging portion 16, which are connected through the grooves to one another. Accordingly, the nucleic acids can be easily extracted on the single base 2.

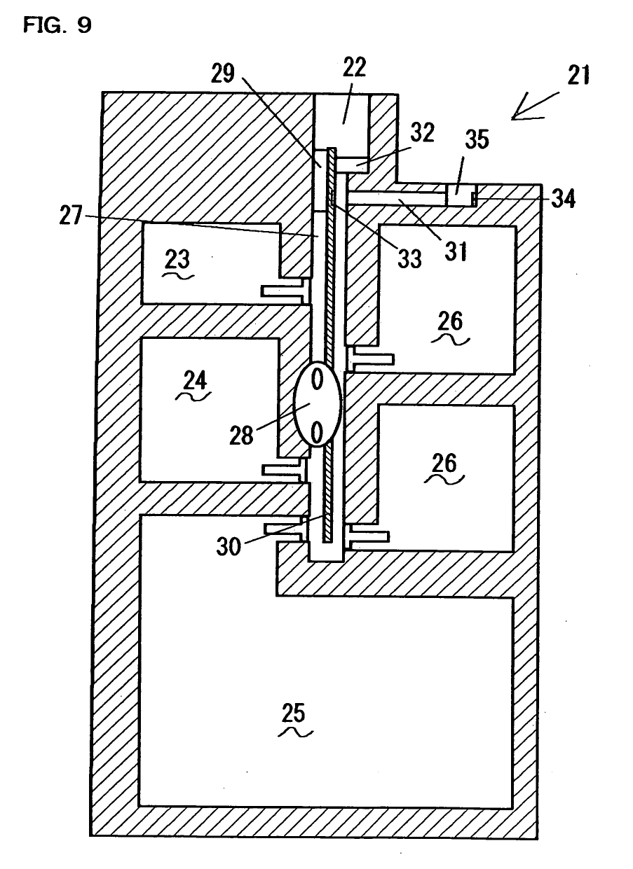

Next, explanation will be given of a second embodiment in accordance with FIG. 9. In a pretreatment device 21 of the second embodiment, liquids circulate vertically so that the nucleic acids are held in the holder and eluted.

The pretreatment device 21 is provided with an introducing portion 22, a holding portion 29, a filter 32, a gel tank 31, a negative electrode 33, a positive electrode 34, an extracting portion 35, an adsorbent liquid storage 23, a wash storages 26, an elute storage 24, and a drain tank 25, which are arranged in order from the upper portion. A circuit 27 connecting the storages to one another is disposed in the center of the pretreatment device 21, and provided with a pump 28. Valves are provided in joints between the circuit 27 and the storages, respectively, so as to control the outflow and inflow of respective liquids. A silica membrane etc. can be used for a holder of the holding portion 29.

In the pretreatment device 21, the specimen is introduced into the introducing portion 22, and then moved into the circuit 27 through the filter 32 by the pump 28. Since the specimen is introduced through the filter 32, badly influential rubbish can be removed from the specimen. The specimen introduced into the circuit 27 is supplied to the holder 29. If necessary, just before supplying the specimen to the holding portion 29, the adsorbent liquid may flows out from the adsorbent liquid storage 23 so that the nucleic acids contained in the specimen are absorbed to the holding portion 29.

After the nucleic acids are held in the holding portion 29 sufficiently, the wash flows out from the wash storage 26 and washes away substances except the nucleic acids held in the holding portion 29. The fluid used for washing is discharged into the drain tank 25. After an end of a certain process of washing, the elute 24 is supplied to the holding portion 29 so as to elute the nucleic acids absorbed to the holding portion 29. The voltage is applied to the negative electrode 33 and the positive electrode 34, whereby the eluted nucleic acids are introduced into the gel tank 31 by means of the electrophoresis. The nucleic acids passing through the gel tank 31 are extracted into the extracting portion 35.

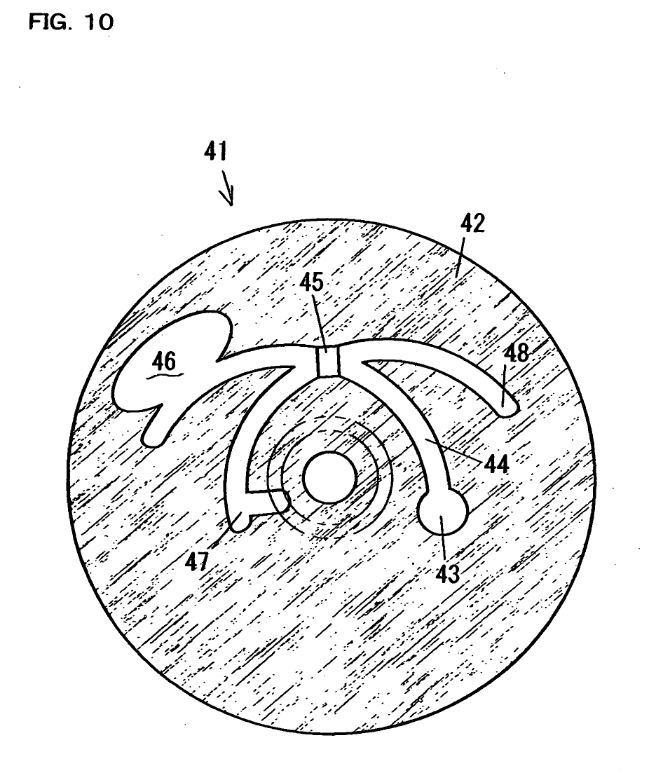

Next, explanation will be given of a third embodiment in accordance with FIG. 10. A pretreatment device 41 of the third embodiment is provided with an introducing portion 43, a specimen supply path 44, an extracting portion 48, a holding portion 45, an elute supply portion 47, and a drain portion 46, which are engraved in a drivable disk 42. A path leading from the introducing portion 43 to the drain portion 46 is constructed in such a way that a distance from the center of the disk 42 to a point along the path is increased as the point approaches the drain portion 46. A path leading from the elute supply portion 47 to the extracting portion 48 is also constructed in such a way that a distance from the center of the disk 42 to a point along the path is increased as the point approaches the extracting portion 48.

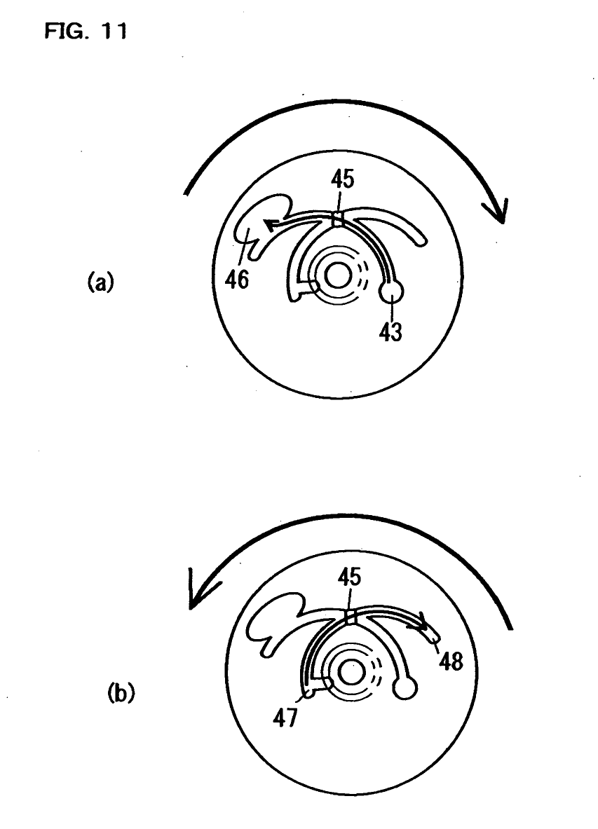

As shown in FIG. 11(a), the disk 42 is clockwise rotated after the specimen is introduced into the introducing portion 43, so the specimen in the introducing portion 43 is supplied to the holding portion 45. At this time, the nucleic acids are absorbed to the holder of the holding portion 45, and components except the nucleic acids are discharged into the drain portion 46. Subsequently, the disk 42 is clockwise rotated after the wash is introduced into the introducing portion 43, so the nucleic acids absorbed to the holding portion 45 can be washed. Successively, the elute is introduced into the elute supply portion 47, and then the disk 42 is rotated counterclockwise as shown in FIG. 11(b), so the elute is supplied from the elute supply portion 47 to the extracting portion 48 through the holding portion 45, thereby supplying the nucleic acids held in the holding portion 45 to the extracting portion 48.

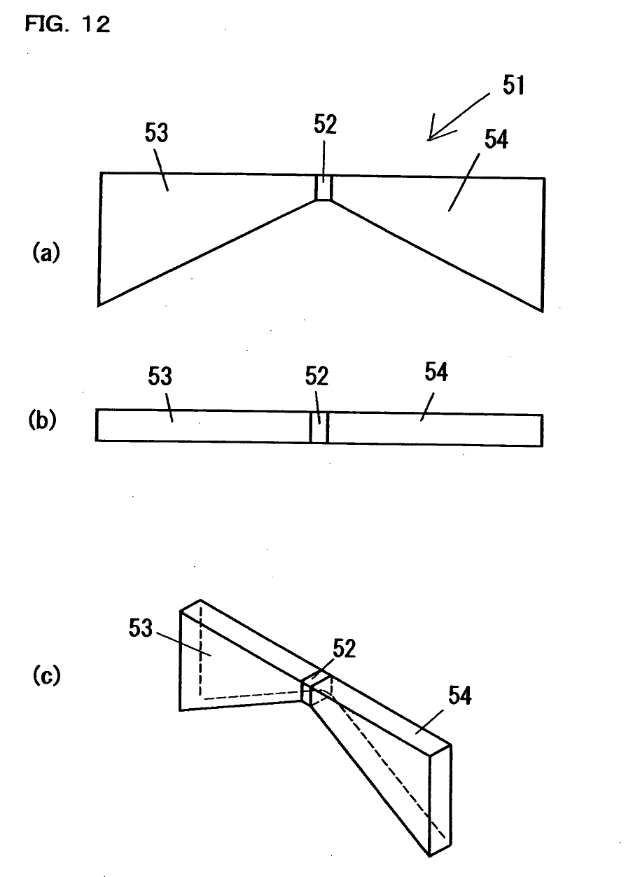

Next, explanation will be given of a fourth embodiment in accordance with FIG. 12. In the fourth embodiment, a pretreatment device 51 comprises a first tank 53, a second tank 54, and a holding portion 52. Bottom faces of the first tank 53 and the second tank 54 are sloped upward to the holding portion 52. Therefore, the specimen is supplied to the first tank 53 and the second tank 54, and then electrophoresed, whereby the nucleic acids in the specimen can be held in the holding portion 52. The holding portion 52 is much smaller than the first tank 53 and the second tank 54 in volume so that the nucleic acids can be easily concentrated in the holding portion 52.

### INDUSTRIAL APPLICABILITY OF THE INVENTION

The device can be provided as a highly sensitive and compact detector due to combination of a function for liberating nucleic acids off from a specimen with a function for extracting and purifying the liberated nucleic acids. Due to the device, the specimen pretreatment can be easily automated and reduced in cost. Additionally, the device can serve as a familiar system for the gene test.

What is claimed is:

1. A device for pretreating a specimen, comprising:
a specimen introducing portion;
a holding portion;
a wash storage;
an elute storage; and
a discharging portion.

2. A device for pretreating a specimen, comprising:
a single base;
a specimen introducing portion for liberating a nucleic acid from the specimen;
a holding portion for holding the nucleic acid;
a wash storage;
an elute storage; and
a discharging portion for discharging liquid,
wherein the specimen introducing portion, the holding portion, the wash storage, the elute storage, and the discharging portion are provided together on the single base.

3. A specimen pretreatment device, comprising:
a base;
a specimen introducing portion for liberating a nucleic acid from the specimen;
a holding portion for holding the nucleic acid;
a discharging portion for discharging a wash;
an extracting portion for extracting the nucleic acid held in the holding portion; and
grooves provided in the base,
wherein, on the base, the holding portion is connected through the grooves to the specimen introducing portion, the discharging portion, and the extracting portion, respectively.

4. A device for pretreating a specimen, comprising:
a base;
a specimen introducing portion for liberating a nucleic acid from the specimen;
a holding portion for holding the nucleic acid;
an extracting portion for collecting the nucleic acid;
a discharging portion for discharging liquid;
an air pump; and
connectors connected to the air pump, wherein the specimen introducing portion, the holding portion, the extracting portion, and the discharging portion are provided together on the base,
wherein the extracting portion and the discharging portion are connected through the respective connectors to the air pump, and
wherein air is inhaled from the connectors so as to control a movement of the liquid on the base.
